(19) United States
(12) Patent Application Publication
(10) Pub. No.: US 2025/0377285 A1
(43) Pub. Date: Dec. 11, 2025

(54) ASSESSMENT OF MICROBIOLOGICALLY INDUCED CORROSION IN PIPELINE

(71) Applicant: SAUDI ARABIAN OIL COMPANY, Dhahran (SA)

(72) Inventors: Abdulrahman Mohammed ALQAHTANI, Khobar (SA); Layan Alaeddin ALSHARIF, Dhahran (SA); Sarah A. AQEEL, Thuwal (SA); Christian Canto MAYA, Dhahran (SA); Faisal AL-ABBAS, Dhahran (SA); Wakeel AHMAD, Dhahran (SA)

(73) Assignee: SAUDI ARABIAN OIL COMPANY, Dhahran (SA)

(21) Appl. No.: 18/734,910

(22) Filed: Jun. 5, 2024

(51) Int. Cl. G01N 17/00; F17D 5/02; G06F 30/28; G06F 113/08; G06F 113/14; G16C 60/00

(52) U.S. Cl. CPC G01N 17/006; F17D 5/02; G06F 30/28; G16C 60/00; G06F 2113/08; G06F 2113/14

(57) ABSTRACT

Methods and systems may be used for mitigation of microbiologically induced corrosion (MIC). For example, a method of mitigation of MIC may include: generating an MIC risk profile for a hydrocarbon pipeline, wherein generating the MIC risk profile comprises: simulating hydraulic flow using a hydraulic model, wherein a hydraulic model input comprises a pipe property, an operational property, a fluid property, or any combination thereof, and wherein a hydraulic model output comprises a hydraulic profile; simulating MIC using an MIC model, wherein an MIC model input comprises the hydraulic profile, a microbial property, or any combination thereof, and wherein an MIC model output comprises biofilm thickness, biofilm density, MIC rate, pitting frequency, or any combination thereof; generating the MIC risk profile based on a likelihood criteria, the MIC model output, or any combination thereof; and analyzing the MIC risk profile in order to calculate an MIC risk score.

### FIELD OF THE DISCLOSURE

The present disclosure relates generally to maintenance of hydrocarbon pipelines and, more particularly, to microbiologically induced corrosion mitigation.

### BACKGROUND OF THE DISCLOSURE

Pipelines provide a cost effective hydrocarbon transport. Pipelines are often made from steel, which makes these pipelines susceptible to corrosion. The corrosion may occur when water or other corrosive materials contact the pipeline, which may occur on the outer surface of the pipeline or on the inner surface of the pipeline due to corrosive materials present in the hydrocarbon being transported. Other types of corrosion may include microbiologically induced corrosion (MIC). MIC occurs when microorganisms, such as bacteria, fungi, or archaea, interact with surfaces (e.g., walls of a pipeline), leading to accelerated corrosion rates. Many methods may be employed to prevent corrosion including surface treatments such as anodization or surface coating.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

Nonlimiting example methods of the present disclosure may include: generating a microbiologically induced corrosion risk profile for a first pipe region of at least one pipe region of a hydrocarbon pipeline configured to carry a hydrocarbon fluid, wherein generating the microbiologically induced corrosion risk profile comprises: simulating hydraulic flow within a first sampling segment within the first pipe region using a hydraulic model, wherein a hydraulic model input comprises a pipe property, an operational property, a fluid property, or any combination thereof, and wherein a hydraulic model output comprises a hydraulic profile; simulating microbiologically induced corrosion within the first sampling segment using a microbiologically induced corrosion model, wherein a microbiologically induced corrosion model input comprises the hydraulic profile, a microbial property, or any combination thereof, and wherein a microbiologically induced corrosion model output comprises biofilm thickness, biofilm density, microbiologically induced corrosion rate, pitting frequency, or any combination thereof; generating the microbiologically induced corrosion risk profile for the first pipe region based on a likelihood criteria, the microbiologically induced corrosion model output, or any combination thereof; and analyzing the microbiologically induced corrosion risk profile in order to calculate a microbiologically induced corrosion risk score for the first pipe region.

Nonlimiting example machine-readable storage mediums of the present disclosure may have stored thereon a computer program for performing the steps of: generating a microbiologically induced corrosion risk profile for a first pipe region of at least one pipe region of a hydrocarbon pipeline configured to carry a hydrocarbon fluid, wherein generating the microbiologically induced corrosion risk profile comprises: simulating hydraulic flow within a first sampling segment within the first pipe region using a hydraulic model, wherein a hydraulic model input comprises a pipe property, an operational property, a fluid property, or any combination thereof, and wherein a hydraulic model output comprises a hydraulic profile; simulating microbiologically induced corrosion within the first sampling segment using a microbiologically induced corrosion model, wherein a microbiologically induced corrosion model input comprises the hydraulic profile, a microbial property, or any combination thereof, and wherein a microbiologically induced corrosion model output comprises biofilm thickness, biofilm density, microbiologically induced corrosion rate, pitting frequency, or any combination thereof; generating the microbiologically induced corrosion risk profile for the first pipe region based on a likelihood criteria, the microbiologically induced corrosion model output, or any combination thereof; and analyzing the microbiologically induced corrosion risk profile in order to calculate a microbiologically induced corrosion risk score for the first pipe region.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 illustrates a diagram of a method for assessing and mitigating microbiologically induced corrosion in accordance with the present disclosure.

FIG. 2 illustrates levels of hierarchy for a nonlimiting example pipeline system of the present disclosure.

FIG. 3 illustrates a diagram of a physical modeling sub-method including a physical microbiologically induced corrosion model in accordance with the present disclosure.

FIGS. 4A and 4B illustrate nonlimiting example graphical user interfaces showing microbiologically induced corrosion risk scores.

FIG. 5 illustrates a nonlimiting example computer system that can be employed to execute one or more embodiments of the present disclosure.

### DETAILED DESCRIPTION

Embodiments of the present disclosure will now be described in detail with reference to the accompanying Figures. Like elements in the various Figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally relate to maintenance of hydrocarbon pipelines and, more particularly, to microbiologically induced corrosion mitigation.

Assessment and mitigation of corrosion, including microbiologically induced corrosion, remains a great cost to operators of hydrocarbon pipelines, particularly in non-scrapable pipelines. A “non-scrapable pipeline” (i.e., a non-piggable pipeline), as used herein, refers to a pipeline that cannot be inspected or cleaned through the use of a “pig” or remote controlled robotic device in the pipeline. Pipelines that are non-scrapable may be more likely to require costly maintenance or replacement due to the reduced monitoring capability. The present disclosure provides a method and system for assessing the microbiologically induced corrosion risk of a pipe region within a hydrocarbon pipeline.

“Hydrocarbon pipeline,” or simply “pipeline” or “pipe” as used herein refers to a pipeline or portion of a pipeline capable of carrying a hydrocarbon (also referred to as “hydrocarbon fluid”) including, but not limited to, dry gas, wet gas, liquid petroleum, oil, methane, ethane, propane, the like, or any combination thereof. It should be noted that the hydrocarbon fluid may comprise additional species, including, but not limited to, water, dissolved solids, the like, or any combination thereof, in any amount.

“Corrosion” as used herein refers to deterioration of a material as a result of contact with a degradative species in its surroundings. In the case of the present disclosure, corrosion may include, but is not limited to, water wetting, sweet corrosion (e.g., CO₂ corrosion), sour corrosion (e.g., H₂S corrosion), microbiologically induced corrosion (MIC), top of the line corrosion (TLC), scale formation, solid accumulation, or any other corrosion method or type known in the art, as well as any combination thereof.

Microbiologically induced corrosion (MIC) may include wherein microbiological organisms (i.e., microorganisms) including, but not limited to, bacteria, fungi, archaea, the like, or any combination thereof, interact with surfaces of the pipeline, leading to increased rates of corrosion thereof. Without being bound by theory, MIC may occur when microorganisms create an electrochemical environment on or near surface(s) of pipelines such that rates of corrosion of the surface(s) increase. Some MIC may occur through formation of one or more biofilms on the surface(s) of a pipeline. Additionally, MIC may occur through means including, but not limited to, for example, acid-producing bacteria (APB), sulfate-reducing bacteria (SRB), the like, or any combination thereof.

Existing methods of modeling corrosion risk (including MIC risk) may involve disparate systems that may individually model hydraulics, fluid flow, and individual pipe corrosion, and may only utilize limited or no historical data concerning pipelines when assessing risk. Additionally, existing systems may be limited to a singular pipe region at one time, necessitating costly manual calculation and limiting the availability of corrosion risk data. Furthermore, existing corrosion risk systems may neglect to include MIC, or may provide limited integration of MIC data.

The present disclosure provides integrated methods and systems for assessing and mitigating MIC utilizing a combination of models and systems to predict MIC within an entire network of pipelines. The present disclosure may allow for increased accuracy of MIC risk predictions due to the combination of multiple models and data sources including historical data as part of MIC risk modeling and assessment. Additionally, the present disclosure may allow for increased precision of MIC risk analysis enabled by dynamic segmentation of pipelines, enabling detailed viewing of risk conditions for many regions within a pipeline network. This risk analysis may also be able to be carried out with greater frequency than a conventional system, as a result of the integrated approach capable of analyzing an entire pipeline network or multiple portions of a pipeline network at once.

Furthermore, the present disclosure also provides MIC risk assessment and mitigation systems and methods that can account for a wide variety of multiphase flow environments and for various types of hydrocarbons.

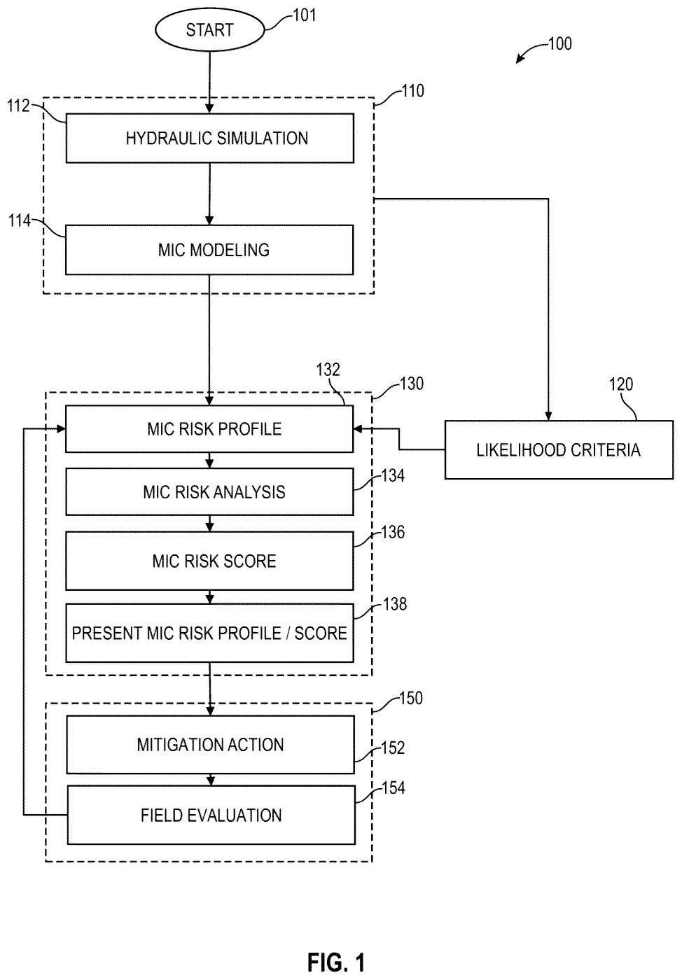

FIG. 1 shows a diagram of a method 100 for assessing and mitigating MIC within a hydrocarbon pipeline according to the present disclosure. A start block 101 may initiate the method. Initial steps of the method may occur as part of the physical modeling sub-method 110. Within the physical modeling sub-method 110, hydraulic simulation 112, and MIC modeling 114 may occur in order to assess individual regions of the pipeline for physical and chemical properties that may influence MIC risk. The physical modeling sub-method 110 may provide data from outputs of individual blocks within the sub-method to likelihood criteria 120 as well as to an MIC risk analysis sub-method 130. The MIC risk analysis sub-method 130 may utilize data outputted from the physical modeling sub-method 110 and from likelihood criteria 120 to assemble an MIC risk profile 132. The data held within the MIC risk profile 132 may be used to analyze the MIC risk for a pipeline as part of MIC risk analysis 134. MIC risk analysis 134 may output an MIC risk score 136 that indicates the risk of MIC for a pipe region or multiple pipe regions. The MIC risk analysis sub-method 130 may further comprise presenting 138 the MIC risk score and the MIC risk profile to the user. Data from the MIC risk profile 132 and MIC risk score 136 may be used to generate and to perform a mitigation action 152 as part of mitigation sub-method 150. The mitigation sub-method 150 may include a field evaluation 154, which may be used to generate data that may be included in the MIC risk profile 132 for further analysis.

It should be noted that the herein described methods and models may also be used in any combination as a part of a system that executes at least a portion of said methods and models according to the present disclosure.

### Pipeline Segmentation

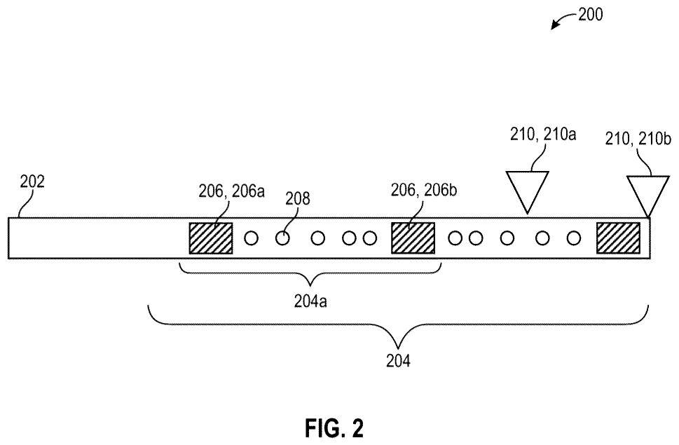

In order to clarify the structure of the methods and systems of the present disclosure, a description of levels of segmentation is provided herein. Within a pipeline there may be multiple levels of segmentation for which data may be provided, generated, or any combination thereof. Methods and systems of the present disclosure may be configured so as to integrate data from various levels of segmentation. FIG. 2 is provided as a nonlimiting example to assist in illustrating the hierarchy of segmentation for any pipeline of interest.

The illustrative pipeline system 200 may comprise a pipeline 202 that may have within it a pipe region 204 (or region). The pipe region 204 may be a region of interest of any size within any pipeline of the present disclosure and may comprise the whole of a pipeline 202. Within the pipe region 204 there may be one or more sub-regions 204a. Within the pipe region 204 there may be one or more sampling segments 206. Between two sampling segments 206a and 206b there may be one or more pipe nodes 208 (or nodes). It should be noted that while 5 nodes are displayed between 206a and 206b in FIG. 2, any number of nodes may exist between two sampling segments. The pipeline 202 may also have one or more points of interest 210 that may be of use in providing discrete data for a specific point along the pipeline 202. Examples of points of interest 210 may include, but are not limited to, a pipe outlet, a wellhead inlet, or any combination thereof. Points of interest 210 may exist within a pipe region 204 of a pipeline 202 (e.g., point of interest 210a) including at a node 208 and may exist at the end of a pipe region 204 or pipeline 202 (e.g., point of interest 210b). Note that some pipelines 202 or some pipe regions 204 may not have any points of interest 210.

Division of levels of hierarchy (e.g., sampling segments, pipe regions, nodes, and the like) may be determined by user input or may be determined dynamically by or based on models of the present disclosure including, but not limited to, a hydraulic model, an MIC model, an (optional) biofilm growth model, the like, or any combination thereof.

Additional levels of hierarchy (e.g., sub-sub-regions within sub-regions) may be used in accordance with the present disclosure. Additional branches, pumps, valves, and other similar features not shown herein may be present along the length of a pipeline in accordance with the present disclosure.

Data used in the MIC risk assessment and mitigation systems and methods in accordance with the present disclosure may be provided for any suitable level of hierarchy within the pipeline system (e.g., for the pipeline as a whole, for a pipe region, for a sampling segment, for a node, for a point of interest, or any combination thereof). For example, pipe diameter may be provided for an entire pipeline. As another example, gas density and flow pattern may be provided, initially, for one or more sampling segments, and, subsequently, gas density and flow pattern may also be provided for one or more nodes between or near the one or more sampling segments.

### Physical Modeling Sub-Method

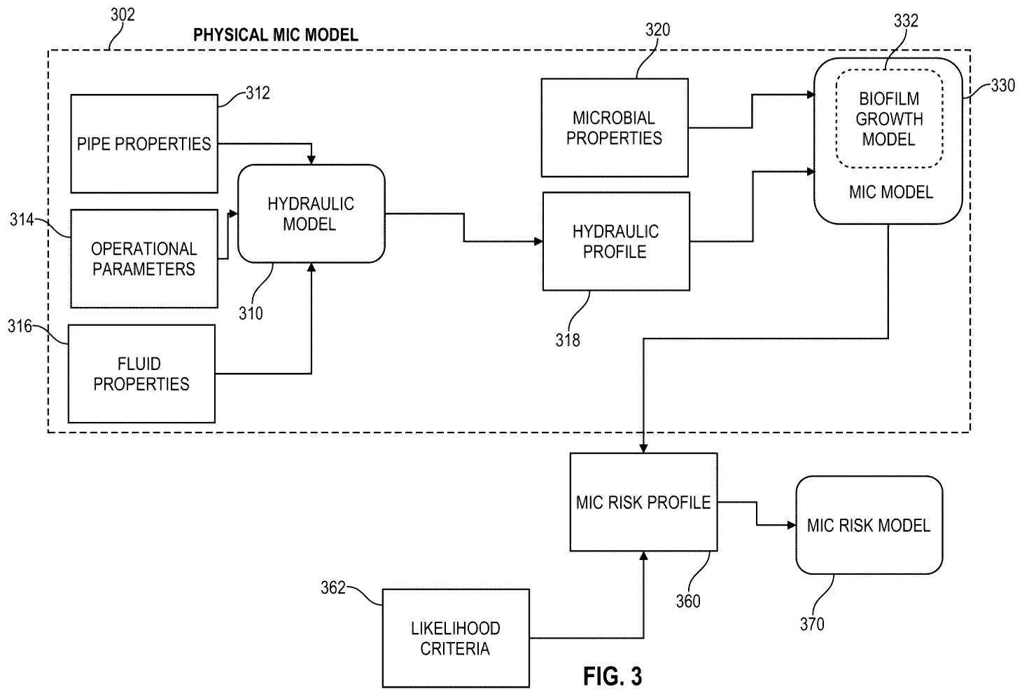

In accordance with the present disclosure, a physical modeling sub-method (equivalent to physical modeling sub-method 110 as depicted in FIG. 1) may be used to model conditions within a pipe region of a hydrocarbon pipeline using a physical MIC model. A diagram of a physical MIC model used as part of physical modeling sub-method is shown in FIG. 3.

The physical MIC model 302 may comprise a hydraulic model 310 and an MIC model 330 (the MIC model 330 optionally including a biofilm growth model 332 therein). The physical MIC model 302 may output data to an MIC risk profile 360. MIC risk profile 360 may subsequently use likelihood criteria 362 to inform an MIC risk model 370.

### Hydraulic Modeling

The hydraulic model 310 may receive hydraulic model inputs that may comprise one or more pipe properties 312, one or more operational parameters 314, one or more fluid properties 316, or any combination thereof. The hydraulic model 310 may dynamically perform an initial segmentation of the pipeline, including, but not limited to, into pipe regions or sampling segments within pipe regions. Initial segmentation may also be conducted by the user in combination with dynamic segmentation or in lieu of dynamic segmentation. Initial segmentation may be based on hydraulic model inputs.

The pipe properties 312 may comprise any pipe property for a pipe region or sampling segment within the pipe region, wherein the pipe property may be relevant for modeling hydraulic flow. The pipe properties 312 may include, but are not limited to, pipe material composition, pipe diameter, pipe elevation, pipe geographic position (e.g., latitude and longitude), pipe interior roughness, pipe thickness, pipe thermal conductivity, the like, or any combination thereof. It should be noted that in the case of discrete properties such as pipe elevation or pipe geographic position, these properties may be provided for a point of interest, which may include an endpoint or midpoint of a pipe region, or any combination thereof.

The operational parameters 314 may comprise any operational properties of the fluid flowing within a pipe region or sampling segment within the pipe region, wherein the operational property may be relevant for modeling hydraulic flow including, but not limited to, sink pressure, liquid phase flowrate, temperature, water cut, gas to liquid ratio, the like, or any combination thereof. As sink pressure is a discrete property it may be localized to a point of interest, such as, for example, to an outlet of a pipe region. Temperature, another discrete property, may be localized to a point of interest, such as, for example, to a wellhead.

The fluid properties 316 may comprise any properties of the fluid within a pipe region or sampling segment within the pipe region, wherein the operational property may be relevant for modeling hydraulic flow including, but not limited to, specific gravity, API gravity, specific heat capacity, viscosity, specific latent heat of vaporization, composition (e.g., mole fraction, mass fraction, volume fraction, and the like), inversion water cut (e.g., emulsion inversion water cut), oil formation volume factor (OFVF), total dissolved solids (TDS), pH, reduction potential, the like, or any combination thereof.

Specific gravity, specific heat capacity, viscosity, and specific latent heat of vaporization may each be for the fluid as a whole, for any constituent component (including oil, gas, water, or the like), or for the fluid as a whole and for any constituent component. Composition may include composition of only primary components within the fluid (e.g., oil, gas, water, or any combination thereof), of only contaminants within the fluid (e.g., CO₂, H₂S, N₂, H₂, CO, and the like), or of primary components and contaminants within the fluid. Water properties including, but not limited to, total dissolved solids (TDS), pH, reduction potential, and the like, may be included for water found within the pipeline and may inform overall water quality.

The hydraulic model 310 may utilize hydraulic model inputs to simulate hydraulic flow within the pipe region including for a sampling segment within a pipe region. The hydraulic model 310 may operate by utilizing laws of chemistry and physics well known in the art to interrelate chemical and physical properties of the pipeline and of species within the pipeline, in order to output data that quantifies properties of and related to fluid flow within the pipeline.

The hydraulic model output may comprise a hydraulic profile 318, as calculated by the hydraulic model 310. Hydraulic profile 318 may be localized to a specific sampling segment within a pipeline of the present disclosure. The hydraulic profile 318 may include, but is not limited to, properties such as in-situ velocity (for a liquid phase, a gas phase, or both), density (of oil, of gas, of water, or any combination thereof), viscosity (of oil, of gas, of water, or any combination thereof), oil-water flow pattern, gas-liquid flow pattern, pressure, the like, or any combination thereof.

### Microbiologically Induced Corrosion (MIC) Modeling

The MIC model 330 may simulate MIC for a pipe region, a pipe node, a sampling segment, or any combination thereof. The MIC model 330 may receive an MIC model input, which may comprise one or more microbial properties 320, the hydraulic profile 318, as well as any hydraulic model input (e.g., the one or more pipe properties 312, the one or more operational parameters 314, the one or more fluid properties 316, or any combination thereof), or any combination thereof.

The one or more microbial properties 320 may include, but are not limited to, microbe type, microbe population, microbe growth data, substrate dependency, biocide efficacy, or localization characteristics.

Microbe type may include, but is not limited to, data regarding species or genus characterization of microbes. Microbe population may include, but is not limited to, data regarding overall microbe population, population of specific genus or species, microbe concentration, the like, or any combination thereof. Microbe growth data may include, but is not limited to, data regarding microbe growth patterns. Substrate dependency may include, but is not limited to, data regarding microbe substrate preferences, effects of substrate on specific microbe species growth, the like, or any combination thereof. Biocide efficacy may include, but is not limited to, data regarding presence of microbe-reducing agents, efficacy of microbe-reducing agents on specific microbe species, the like, or any combination thereof. Localization characteristics may include, but is not limited to, data regarding planktonic tendency and/or sessile tendency of specific microbe species.

The microbial properties 320 may be localized to a pipe region, to a sampling segment, or to a node therebetween. Microbial properties 320 may be derived from various sources including, but not limited to, published values, experimental data, the like, or any combination thereof. Experimental data may include, but is not limited to, field testing, laboratory testing, the like, or any combination thereof.

The MIC model 330 may operate by utilizing laws of biology, chemistry, and/or physics well known in the art to interrelate biological, chemical, and/or physical properties of the pipeline and of species within the pipeline, in order to output data that quantifies MIC progression and related properties within the pipeline, including, but not limited to, for example, pitting, MIC rate, the like, or any combination thereof.

The MIC model 330 may, optionally, include therein biofilm growth model 332. Biofilm growth model 332 may operate by utilizing laws of biology, chemistry, and/or physics well known in the art to interrelate biological, chemical, and/or physical properties in order to quantify formation of microbes into a biofilm, if present. It should be noted that in some embodiments biofilm growth model 332 may or may not be used within MIC model 330.

“Biofilm,” and grammatical variations thereof, as used herein, refers to a syntrophic layer of microorganisms formed on a surface (e.g., a surface of a pipeline).

The MIC model 330 may produce one or more MIC model outputs based on simulating MIC that may include, but is not limited to, biofilm thickness, biofilm density, MIC rate, pitting frequency, the like, or any combination thereof.

It should be noted that portions of or the whole of the physical MIC model 302, including the hydraulic model 310, MIC model 330, and biofilm growth model 332, may be iterated one time or more than one time in order to, for example, increase precision of calculations or perform calculations for additional pipe regions or nodes. Data generated in one iteration may be used by any component of the physical MIC model 302 in a subsequent iteration.

### MIC Risk Profile

After a single or multiple iterations of the physical MIC model 302, the physical MIC model 302 subsequently outputs data to MIC risk profile 360 (equivalent to MIC risk profile 132 in FIG. 1). The MIC risk profile 360 may comprise a set of data that may assist in calculating the estimated risk of MIC. This data may include inputs, outputs, or inputs and outputs of any of the models including the hydraulic model 310, MIC model 330, and biofilm growth model 332. MIC risk profile 360 may include MIC properties including, but not limited to, for example, corrosion rates. It should be noted that MIC properties may be localized to a pipe region, to a sampling segment, or to a node therebetween.

The MIC risk profile 132, 360 may additionally include likelihood criteria (120 in FIGS. 1 and 362 in FIG. 3).

Likelihood criteria 120, 362 comprise any data that may support calculation of MIC risk. Likelihood criteria 120, 362 may be based on any suitable source including calculation, field analysis, lab experimentation, the like, or any combination thereof.

Likelihood criteria 120, 362 may comprise historical data for the pipe region, or one or more points of interest within the pipe region and may include production history, leak history, or any combination thereof.

Production history may comprise current operational data, past operational data, or a combination thereof for a pipeline or pipe region as well as the fluid flowing therein and may include, but is not limited to, operational uptime, flowrate (e.g., mass flowrate, volume flowrate), fluid types (e.g., fluid composition, including water cut), fluid velocity, fluid corrosivity, microbial load, corrosion inhibitor use (including corrosion inhibitor composition, corrosion inhibitor quantity, the like, or any combination thereof), the like, or any combination thereof. Production history data may be provided in discrete form (for one or more specific individual hours, days, months, years, or longer) or may be provided in summary form (e.g., 3100 hours of 94.9% methane flow with 3.2% water content). Production history data may inform the extent to which a pipeline has been operated intermittently, which, without being bound by theory, may increase the likelihood of corrosion, including MIC, occurring.

Leak history may comprise data on specific leaks throughout the history of a pipeline and may be localized to a point of interest where the leak occurred or continues to occur or may be localized to a pipe region. Leak history may include, but is not limited to, leak physical geometry (e.g., 1 cm diameter leak), leak flow size (e.g., 5 barrel per day leak), leak location (including approximate localization to a pipe region or precise localization to a point of interest where the leak occurred).

Likelihood criteria 120, 362 may further comprise pipe coating composition, pipe coating application history (e.g., when a pipe coating was applied), and pipe coating location(s). Likelihood criteria 120, 362 may further comprise one or more scraping compliance metrics (e.g., scraping frequency, scraping history, scraping type, and the like).

Likelihood criteria 120, 362 may further comprise biocide use data, including, but not limited to, biocide use concentration, biocide type, biocide use frequency, the like, or any combination thereof.

### MIC Risk Sub-Method

Referring back to FIG. 1, following the physical modeling sub-method 110, the system may carry out an MIC risk analysis sub-method 130, which may comprise the generation of an MIC risk profile 132 and an MIC risk analysis 134 that may produce an MIC risk score 136. The sub-method may further comprise wherein the MIC risk profile, the MIC risk score, or both are presented 138 to the user.

### MIC Risk Analysis and MIC Score Generation

The MIC risk profile 132, 360 is utilized to conduct an MIC risk analysis 134 for a pipe region. The MIC risk analysis 134 statistically analyzes one or more factors (based on data from the MIC risk profile 132, 360), which may predict the risk of MIC by comparing the one or more factors to known indicators of MIC or lack thereof.

The MIC risk analysis 134 may output an MIC risk score 136. The MIC risk score 136 may comprise a numerical score for a pipe region (or sampling segment, or pipe node), which quantifies the level of MIC risk. The MIC risk score 136 may comprise an integer on a scale (e.g., 0 to 10 or 1 to 100) wherein one numerical end of the scale indicates higher MIC risk (e.g., a higher probability of failure or degraded operation due to MIC) and the other end of the scale indicates lower MIC risk (e.g., a lower probability of failure or degraded operation due to MIC).

It should be noted that a unique MIC risk score 136 may be generated for each of multiple regions (e.g., 4 regions may each have an MIC risk score) within a pipeline, enabling quantification of varying levels of MIC risk along a pipeline.

### Presentation of MIC Data

The MIC risk profile, the MIC risk score, or both may be presented 138 to the user. Presenting 138 the MIC risk profile, the MIC risk score, or both may comprise presenting in a graphical user interface (GUI). The GUI may comprise a geographic map in which representations of one or more pipelines and constituent pipe regions are displayed in such a manner that the one or more pipelines are localized to one or more locations on the geographic map. A color code may overlay the representations of the one or more pipe regions and the color code may correspond to the MIC risk score for that pipe region. The color code may be such that a certain color or colors correspond to the MIC risk score based on threshold value(s), which may be adjusted by the user within the graphical user interface or may be preset by the system. The GUI may allow the user to zoom in and to zoom out in order to view the representations of the pipeline or pipe region in greater detail. The GUI may also allow the user to view data from the MIC risk profile that is localized to the representation of the pipeline or pipe region as displayed. The GUI may also allow the user to view additional non-localized data of the MIC risk profile including the whole corrosion risk profile. The GUI may also display a representation of a point of interest. The GUI may also display a representation of a pipe node. The GUI may also display additional data from the MIC risk profile localized to the representation of the point of interest, the representation of the pipe node, or to a combination thereof.

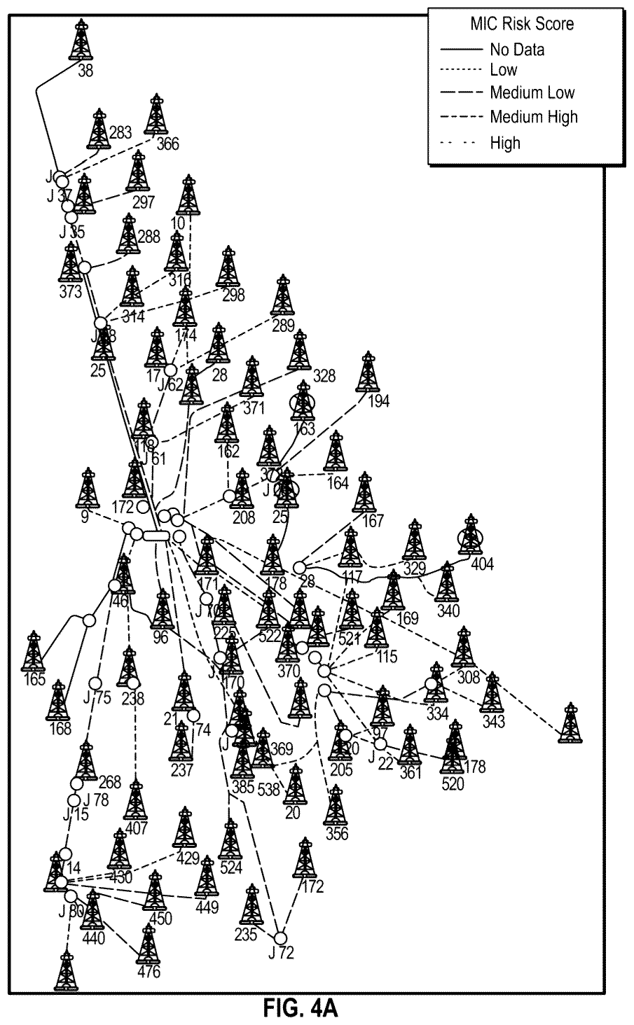

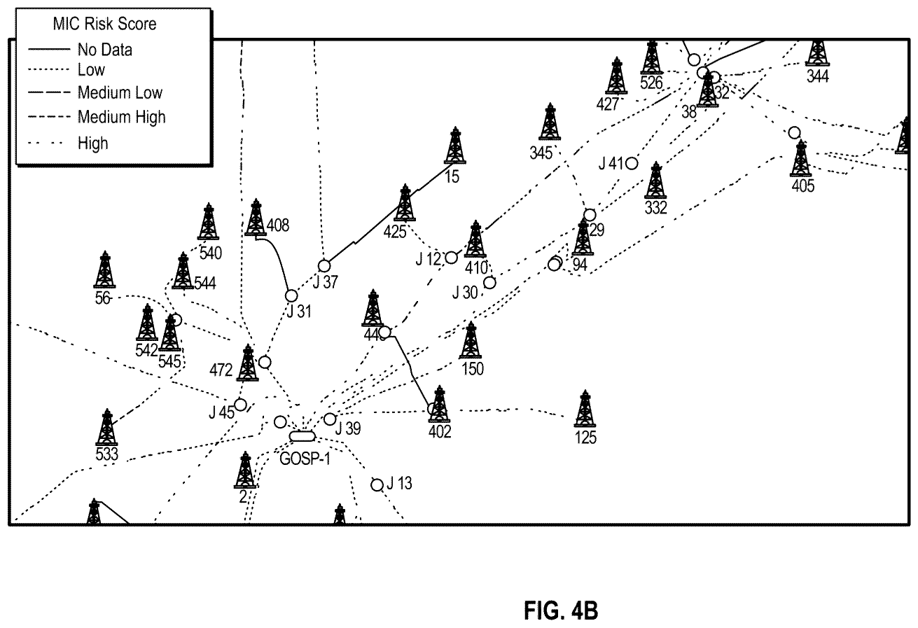

As a nonlimiting example, FIGS. 4A and 4B each show a GUI that displays representations of one or more pipelines and constituent pipe regions on a geographic map. FIGS. 4A and 4B show the MIC risk score overlaid as a color code on the geographic map.

The GUI may enable a user to visually identify one or more MIC risk clusters. An MIC risk cluster may comprise wherein more than one pipe region located in proximity to another may have an MIC risk score that indicates a higher probability of MIC. Identification of MIC risk clusters may be of particular importance to prioritizing various pipe regions for MIC mitigation.

### Mitigation Action

The methods and systems of the present disclosure may further comprise performing one or more mitigation actions for a pipe region based on the MIC risk profile, the MIC risk score, or both. The one or more mitigation actions may include, but are not limited to, conducting a field evaluation, repairing or rehabilitating the pipe region, replacing the pipe region, adjusting use of a biocide, generating an MIC mitigation plan for the pipe region, the like, or any combination thereof.

Field evaluation may comprise pipeline health monitoring through any suitable means known in the art including, but not limited to, online sampling, materials testing, visual inspection, or any combination thereof.

Adjusting use of a biocide may include increasing or decreasing use of a biocide in in a pipeline or pipe region, or may include selection of a specific biocide for use in a pipeline or pipe region.

The one or more mitigation actions may be carried out for a pipeline or pipe region. A single mitigation action for a pipeline or pipe region may be applied to one or more pipelines or one or more pipe regions (e.g., a mitigation in a singular area of a pipe region may serve as mitigation for an entire pipeline).

It should be noted that the one or more mitigation actions may be performed by a person, a machine, or any combination thereof.

### Computer System

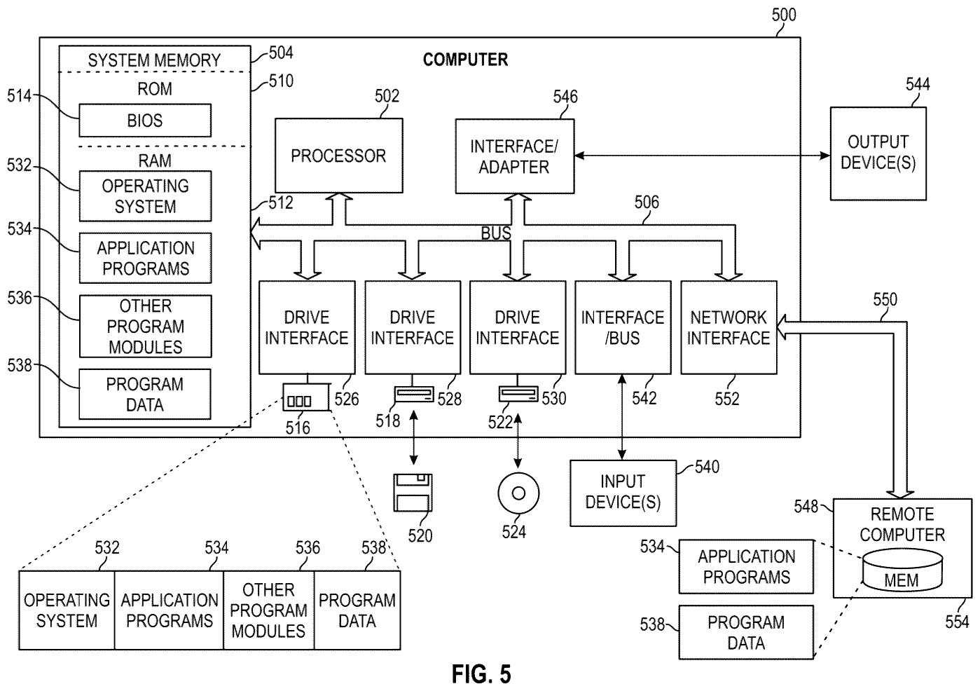

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 5. Furthermore, portions of the embodiments may be a computer program product on a computer-usable storage medium having computer-readable program code on the medium. Any non-transitory, tangible storage media possessing structure may be utilized including, but not limited to, static and dynamic storage devices, hard disks, optical storage devices, and magnetic storage devices, but excludes any medium that is not eligible for patent protection under 35 U.S.C. § 101 (such as a propagating electrical or electromagnetic signals per se). As an example and not by way of limitation, computer-readable storage media may include a semiconductor-based circuit or device or other IC (such, as for example, a field-programmable gate array (FPGA) or an application-specific integrated circuit (ASIC)), a hard disk, a hard disk drive (HDD), a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. A computer-readable non-transitory storage medium may be volatile, nonvolatile, or a combination of volatile and non-volatile, as appropriate.

Certain embodiments have also been described herein with reference to block illustrations of methods, systems, and computer program products. It will be understood that blocks and/or combinations of blocks in the illustrations, as well as methods or steps or acts or processes described herein, can be implemented by a computer program comprising a routine of set instructions stored in a machine-readable storage medium as described herein. These instructions may be provided to one or more processors of a general purpose computer, special purpose computer, or other programmable data processing apparatus (or a combination of devices and circuits) to produce a machine, such that the instructions of the machine, when executed by the processor, implement the functions specified in the block or blocks, or in the acts, steps, methods and processes described herein.

These processor-executable instructions may also be stored in computer-readable memory that can direct a computer or other programmable data processing apparatus to function in a particular manner, such that the instructions stored in the computer-readable memory result in an article of manufacture including instructions that implement the function specified. The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide steps for implementing the functions specified in the flowchart block or blocks.

In this regard, FIG. 5 illustrates one example of a computer system 500 that can be employed to execute one or more embodiments of the present disclosure. Computer system 500 can be implemented on one or more general purpose networked computer systems, embedded computer systems, routers, switches, server devices, client devices, various intermediate devices/nodes or standalone computer systems. Additionally, computer system 500 can be implemented on various mobile clients such as, for example, a personal digital assistant (PDA), laptop computer, pager, and the like, provided it includes sufficient processing capabilities.

Computer system 500 includes processing unit 502, system memory 504, and system bus 506 that couples various system components, including the system memory 504, to processing unit 502. Dual microprocessors and other multi-processor architectures also can be used as processing unit 502. System bus 506 may be any of several types of bus structure including a memory bus or memory controller, a peripheral bus, and a local bus using any of a variety of bus architectures. System memory 504 includes read only memory (ROM) 510 and random access memory (RAM) 512. A basic input/output system (BIOS) 514 can reside in ROM 510 containing the basic routines that help to transfer information among elements within computer system 500.

Computer system 500 can include a hard disk drive 516, magnetic disk drive 518, e.g., to read from or write to removable disk 520, and an optical disk drive 522, e.g., for reading CD-ROM disk 524 or to read from or write to other optical media. Hard disk drive 516, magnetic disk drive 518, and optical disk drive 522 are connected to system bus 506 by a hard disk drive interface 526, a magnetic disk drive interface 528, and an optical drive interface 530, respectively. The drives and associated computer-readable media provide nonvolatile storage of data, data structures, and computer-executable instructions for computer system 500. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and described herein.

A number of program modules may be stored in drives and RAM 512, including operating system 532, one or more application programs 534, other program modules 536, and program data 538. In some examples, the application programs 534 can include the physical modeling sub-method 110, the hydraulic model 310, the biofilm growth model 332, the MIC model 330, the MIC risk analysis sub-method 130, the mitigation sub-method 150, and the program data 538 can include pipe properties 312, operational parameters 314, fluid properties 316, hydraulic profile 318, microbial properties 320, likelihood criteria 120 or 362, MIC risk profile 132 or 360, MIC risk score 136, and mitigation action 152. The application programs 534 and program data 538 can include functions and methods programmed to evaluate, quantify, or mitigate (or any combination thereof) the corrosion risk of a pipeline or pipe region within a pipeline, such as shown and described herein.

A user may enter commands and information into computer system 500 through one or more input devices 540, such as a pointing device (e.g., a mouse, touch screen), keyboard, microphone, joystick, game pad, scanner, and the like. For instance, the user can employ input device 540 to edit or modify pipe properties 312, operational parameters 314, fluid properties 316, hydraulic profile 318, microbial properties 320, likelihood criteria 120 or 362, MIC risk profile 132 or 360, MIC risk score 136, mitigation action 152, or any combination thereof. As another example, the user may also employ input device 540 to view data from the MIC risk profile 132 or 360, the MIC risk score 136, or any combination thereof. These and other input devices 540 are often connected to processing unit 502 through a corresponding port interface 542 that is coupled to the system bus, but may be connected by other interfaces, such as a parallel port, serial port, or universal serial bus (USB). One or more output devices 544 (e.g., display, a monitor, printer, projector, or other type of displaying device) are also connected to system bus 506 via interface 546, such as a video adapter.

Computer system 500 may operate in a networked environment using logical connections to one or more remote computers, such as remote computer 548. Remote computer 548 may be a workstation, computer system, router, peer device, or other common network node, and typically includes many or all of the elements described relative to computer system 500. The logical connections, schematically indicated at 550, can include a local area network (LAN) and/or a wide area network (WAN), or a combination of these, and can be in a cloud-type architecture, for example configured as private clouds, public clouds, hybrid clouds, and multi-clouds. When used in a LAN networking environment, computer system 500 can be connected to the local network through a network interface or adapter 552. When used in a WAN networking environment, computer system 500 can include a modem, or can be connected to a communications server on the LAN. The modem, which may be internal or external, can be connected to system bus 506 via an appropriate port interface. In a networked environment, application programs 534 or program data 538 depicted relative to computer system 500, or portions thereof, may be stored in a remote memory storage device 554.

### Additional Embodiments

Embodiments disclosed herein include:

A. Generating a microbiologically induced corrosion risk profile for a first pipe region of at least one pipe region of a hydrocarbon pipeline configured to carry a hydrocarbon fluid, wherein generating the microbiologically induced corrosion risk profile comprises: simulating hydraulic flow within a first sampling segment within the first pipe region using a hydraulic model, wherein a hydraulic model input comprises a pipe property, an operational property, a fluid property, or any combination thereof, and wherein a hydraulic model output comprises a hydraulic profile; simulating microbiologically induced corrosion within the first sampling segment using a microbiologically induced corrosion model, wherein a microbiologically induced corrosion model input comprises the hydraulic profile, a microbial property, or any combination thereof, and wherein a microbiologically induced corrosion model output comprises biofilm thickness, biofilm density, microbiologically induced corrosion rate, pitting frequency, or any combination thereof; generating the microbiologically induced corrosion risk profile for the first pipe region based on a likelihood criteria, the microbiologically induced corrosion model output, or any combination thereof; and analyzing the microbiologically induced corrosion risk profile in order to calculate a microbiologically induced corrosion risk score for the first pipe region.

B. A method comprising the steps of Embodiment A.

C. A machine-readable storage medium having stored thereon a computer program for performing the steps of Embodiment A.

Each of Embodiments A through C may have one or more of the following additional elements in any combination:
Element 1: performing at least one mitigation action for the first pipe region based on the microbiologically induced corrosion risk score, the microbiologically induced corrosion risk profile, or any combination thereof.
Element 2: wherein performing the at least one mitigation action for the first pipe region comprises: conducting a field evaluation of the first pipe region, rehabilitating the first pipe region, replacing at least a portion of the first pipe region, generating a corrosion mitigation plan for the first pipe region, or any combination thereof.
Element 3: wherein the microbial property comprises microbe type, microbe population, microbe growth data, substrate dependency, biocide efficacy, localization characteristics, or any combination thereof.
Element 4: wherein simulating microbiologically induced corrosion within the first sampling segment using a microbiologically induced corrosion model further comprises simulating biofilm growth using a biofilm growth model, wherein a biofilm growth model output comprises the biofilm thickness, the biofilm density, or any combination thereof.
Element 5: further comprising displaying the microbiologically induced corrosion risk score and, optionally, the microbiologically induced corrosion risk profile, in a graphical user interface, wherein the graphical user interface comprises a geographic map that comprises a representation of the first pipe region localized to one or more locations on the geographic map, and wherein the microbiologically induced corrosion risk score is displayed as a color code overlaid on the representation of the first pipe region.
Element 6: further comprising identifying a microbiologically induced corrosion risk cluster using the graphical user interface.
Element 7: wherein the hydraulic profile comprises: a liquid phase in-situ velocity, a gas phase in-situ velocity, an oil density, a gas density, a water density, an oil viscosity, a gas viscosity, a water viscosity, an oil-water flow pattern, a gas-liquid flow pattern, a pressure, or any combination thereof.
Element 8: wherein the likelihood criteria comprises: a production history, a leak history, a pipe coating composition, a pipe coating application history, a pipe coating location, a scraping compliance metric, biocide use data, or any combination thereof.
Element 9: wherein the hydrocarbon pipeline comprises a dry gas pipeline, a wet gas pipeline, a liquid petroleum pipeline, or a multiphase pipeline.
Element 10: wherein calculating the microbiologically induced corrosion risk score comprises performing a statistical analysis using the microbiologically induced corrosion risk profile.

By way of nonlimiting example, exemplary combinations of Elements applicable to A through C include: 1 and 2, 5 and 6, 1-3, 1-4, 1-5, 1-6, 5-7, 5-8, 5-9, 5-10, 1 and 10, 1 and 5-6, 1-2 and 5-6.

Further exemplary embodiments may include:
Clause 1. A method comprising: generating a microbiologically induced corrosion risk profile for a first pipe region of at least one pipe region of a hydrocarbon pipeline configured to carry a hydrocarbon fluid, wherein generating the microbiologically induced corrosion risk profile comprises: simulating hydraulic flow within a first sampling segment within the first pipe region using a hydraulic model, wherein a hydraulic model input comprises a pipe property, an operational property, a fluid property, or any combination thereof, and wherein a hydraulic model output comprises a hydraulic profile; simulating microbiologically induced corrosion within the first sampling segment using a microbiologically induced corrosion model, wherein a microbiologically induced corrosion model input comprises the hydraulic profile, a microbial property, or any combination thereof, and wherein a microbiologically induced corrosion model output comprises biofilm thickness, biofilm density, microbiologically induced corrosion rate, pitting frequency, or any combination thereof; generating the microbiologically induced corrosion risk profile for the first pipe region based on a likelihood criteria, the microbiologically induced corrosion model output, or any combination thereof; and analyzing the microbiologically induced corrosion risk profile in order to calculate a microbiologically induced corrosion risk score for the first pipe region.
Clause 2. The method of Clause 1, further comprising: performing at least one mitigation action for the first pipe region based on the microbiologically induced corrosion risk score, the microbiologically induced corrosion risk profile, or any combination thereof.
Clause 3. The method of Clause 1 or 2, wherein performing the at least one mitigation action for the first pipe region comprises: conducting a field evaluation of the first pipe region, rehabilitating the first pipe region, replacing at least a portion of the first pipe region, generating a corrosion mitigation plan for the first pipe region, or any combination thereof.
Clause 4. The method of any one of Clauses 1-3, wherein the microbial property comprises microbe type, microbe population, microbe growth data, substrate dependency, biocide efficacy, localization characteristics, or any combination thereof.
Clause 5. The method of any one of Clauses 1-4, wherein simulating microbiologically induced corrosion within the first sampling segment using a microbiologically induced corrosion model further comprises simulating biofilm growth using a biofilm growth model, wherein a biofilm growth model output comprises the biofilm thickness, the biofilm density, or any combination thereof.
Clause 6. The method of any one of Clauses 1-5, further comprising displaying the microbiologically induced corrosion risk score and, optionally, the microbiologically induced corrosion risk profile, in a graphical user interface, wherein the graphical user interface comprises a geographic map that comprises a representation of the first pipe region localized to one or more locations on the geographic map, and wherein the microbiologically induced corrosion risk score is displayed as a color code overlaid on the representation of the first pipe region.
Clause 7. The method of Clause 6, further comprising identifying a microbiologically induced corrosion risk cluster using the graphical user interface.
Clause 8. The method of any one of Clauses 1-7, wherein the hydraulic profile comprises: a liquid phase in-situ velocity, a gas phase in-situ velocity, an oil density, a gas density, a water density, an oil viscosity, a gas viscosity, a water viscosity, an oil-water flow pattern, a gas-liquid flow pattern, a pressure, or any combination thereof.
Clause 9. The method of any one of Clauses 1-8, wherein the likelihood criteria comprises: a production history, a leak history, a pipe coating composition, a pipe coating application history, a pipe coating location, a scraping compliance metric, biocide use data, or any combination thereof.
Clause 10. The method of any one of Clauses 1-9, wherein the hydrocarbon pipeline comprises a dry gas pipeline, a wet gas pipeline, a liquid petroleum pipeline, or a multiphase pipeline.
Clause 11. The method of any one of Clauses 1-10, wherein calculating the microbiologically induced corrosion risk score comprises performing a statistical analysis using the microbiologically induced corrosion risk profile.
Clause 12. A machine-readable storage medium having stored thereon a computer program for performing the steps of: generating a microbiologically induced corrosion risk profile for a first pipe region of at least one pipe region of a hydrocarbon pipeline configured to carry a hydrocarbon fluid, wherein generating the microbiologically induced corrosion risk profile comprises: simulating hydraulic flow within a first sampling segment within the first pipe region using a hydraulic model, wherein a hydraulic model input comprises a pipe property, an operational property, a fluid property, or any combination thereof, and wherein a hydraulic model output comprises a hydraulic profile; simulating microbiologically induced corrosion within the first sampling segment using a microbiologically induced corrosion model, wherein a microbiologically induced corrosion model input comprises the hydraulic profile, a microbial property, or any combination thereof, and wherein a microbiologically induced corrosion model output comprises biofilm thickness, biofilm density, microbiologically induced corrosion rate, pitting frequency, or any combination thereof; generating the microbiologically induced corrosion risk profile for the first pipe region based on a likelihood criteria, the microbiologically induced corrosion model output, or any combination thereof; and analyzing the microbiologically induced corrosion risk profile in order to calculate a microbiologically induced corrosion risk score for the first pipe region.
Clause 13. The machine-readable storage medium of Clause 12, wherein the steps further comprise: performing at least one mitigation action for the first pipe region based on the microbiologically induced corrosion risk score, the microbiologically induced corrosion risk profile, or any combination thereof.
Clause 14. The machine-readable storage medium of Clause 12 or 13, wherein performing the at least one mitigation action for the first pipe region comprises: conducting a field evaluation of the first pipe region, rehabilitating the first pipe region, replacing at least a portion of the first pipe region, generating a corrosion mitigation plan for the first pipe region, or any combination thereof.
Clause 15. The machine-readable storage medium of any one of Clauses 12-14, wherein the microbial property comprises microbe type, microbe population, microbe growth data, substrate dependency, biocide efficacy, localization characteristics, or any combination thereof.
Clause 16. The machine-readable storage medium of any one of Clauses 12-15, wherein simulating microbiologically induced corrosion within the first sampling segment using a microbiologically induced corrosion model further comprises simulating biofilm growth using a biofilm growth model, wherein a biofilm growth model output comprises the biofilm thickness, the biofilm density, or any combination thereof.
Clause 17. The machine-readable storage medium of any one of Clauses 12-16, wherein the steps further comprise: displaying the microbiologically induced corrosion risk score and, optionally, the microbiologically induced corrosion risk profile, in a graphical user interface, wherein the graphical user interface comprises a geographic map that comprises a representation of the first pipe region localized to one or more locations on the geographic map, and wherein the microbiologically induced corrosion risk score is displayed as a color code overlaid on the representation of the first pipe region.
Clause 18. The machine-readable storage medium of any one of Clauses 12-17, wherein the hydraulic profile comprises: a liquid phase in-situ velocity, a gas phase in-situ velocity, an oil density, a gas density, a water density, an oil viscosity, a gas viscosity, a water viscosity, an oil-water flow pattern, a gas-liquid flow pattern, a pressure, or any combination thereof.
Clause 19. The machine-readable storage medium of any one of Clauses 12-18, wherein the likelihood criteria comprises: a production history, a leak history, a pipe coating composition, a pipe coating application history, a pipe coating location, a scraping compliance metric, biocide use data, or any combination thereof.
Clause 20. The machine-readable storage medium of any one of Clauses 12-19, wherein calculating the microbiologically induced corrosion risk score comprises performing a statistical analysis using the microbiologically induced corrosion risk profile.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

What claimed is:

1. A method comprising:
generating a microbiologically induced corrosion risk profile for a first pipe region of at least one pipe region of a hydrocarbon pipeline configured to carry a hydrocarbon fluid, wherein generating the microbiologically induced corrosion risk profile comprises:
simulating hydraulic flow within a first sampling segment within the first pipe region using a hydraulic model, wherein a hydraulic model input comprises a pipe property, an operational property, a fluid property, or any combination thereof, and wherein a hydraulic model output comprises a hydraulic profile;
simulating microbiologically induced corrosion within the first sampling segment using a microbiologically induced corrosion model, wherein a microbiologically induced corrosion model input comprises the hydraulic profile, a microbial property, or any combination thereof, and wherein a microbiologically induced corrosion model output comprises biofilm thickness, biofilm density, microbiologically induced corrosion rate, pitting frequency, or any combination thereof;
generating the microbiologically induced corrosion risk profile for the first pipe region based on a likelihood criteria, the microbiologically induced corrosion model output, or any combination thereof; and
analyzing the microbiologically induced corrosion risk profile in order to calculate a microbiologically induced corrosion risk score for the first pipe region.

2. The method of claim 1, further comprising:
performing at least one mitigation action for the first pipe region based on the microbiologically induced corrosion risk score, the microbiologically induced corrosion risk profile, or any combination thereof.

3. The method of claim 2, wherein performing the at least one mitigation action for the first pipe region comprises: conducting a field evaluation of the first pipe region, rehabilitating the first pipe region, replacing at least a portion of the first pipe region, generating a corrosion mitigation plan for the first pipe region, or any combination thereof.

4. The method of claim 1, wherein the microbial property comprises microbe type, microbe population, microbe growth data, substrate dependency, biocide efficacy, localization characteristics, or any combination thereof.

5. The method of claim 1, wherein simulating microbiologically induced corrosion within the first sampling segment using a microbiologically induced corrosion model further comprises simulating biofilm growth using a biofilm growth model, wherein a biofilm growth model output comprises the biofilm thickness, the biofilm density, or any combination thereof.

6. The method of claim 1, further comprising displaying the microbiologically induced corrosion risk score and, optionally, the microbiologically induced corrosion risk profile, in a graphical user interface, wherein the graphical user interface comprises a geographic map that comprises a representation of the first pipe region localized to one or more locations on the geographic map, and wherein the microbiologically induced corrosion risk score is displayed as a color code overlaid on the representation of the first pipe region.

7. The method of claim 6, further comprising identifying a microbiologically induced corrosion risk cluster using the graphical user interface.

8. The method of claim 1, wherein the hydraulic profile comprises: a liquid phase in-situ velocity, a gas phase in-situ velocity, an oil density, a gas density, a water density, an oil viscosity, a gas viscosity, a water viscosity, an oil-water flow pattern, a gas-liquid flow pattern, a pressure, or any combination thereof.

9. The method of claim 1, wherein the likelihood criteria comprises: a production history, a leak history, a pipe coating composition, a pipe coating application history, a pipe coating location, a scraping compliance metric, biocide use data, or any combination thereof.

10. The method of claim 1, wherein the hydrocarbon pipeline comprises a dry gas pipeline, a wet gas pipeline, a liquid petroleum pipeline, or a multiphase pipeline.

11. The method of claim 1, wherein calculating the microbiologically induced corrosion risk score comprises performing a statistical analysis using the microbiologically induced corrosion risk profile.

12. A machine-readable storage medium having stored thereon a computer program for performing the steps of:
generating a microbiologically induced corrosion risk profile for a first pipe region of at least one pipe region of a hydrocarbon pipeline configured to carry a hydrocarbon fluid, wherein generating the microbiologically induced corrosion risk profile comprises:
simulating hydraulic flow within a first sampling segment within the first pipe region using a hydraulic model, wherein a hydraulic model input comprises a pipe property, an operational property, a fluid property, or any combination thereof, and wherein a hydraulic model output comprises a hydraulic profile;
simulating microbiologically induced corrosion within the first sampling segment using a microbiologically induced corrosion model, wherein a microbiologically induced corrosion model input comprises the hydraulic profile, a microbial property, or any combination thereof, and wherein a microbiologically induced corrosion model output comprises biofilm thickness, biofilm density, microbiologically induced corrosion rate, pitting frequency, or any combination thereof;
generating the microbiologically induced corrosion risk profile for the first pipe region based on a likelihood criteria, the microbiologically induced corrosion model output, or any combination thereof; and
analyzing the microbiologically induced corrosion risk profile in order to calculate a microbiologically induced corrosion risk score for the first pipe region.

13. The machine-readable storage medium of claim 12, wherein the steps further comprise:
performing at least one mitigation action for the first pipe region based on the microbiologically induced corrosion risk score, the microbiologically induced corrosion risk profile, or any combination thereof.

14. The machine-readable storage medium of claim 13, wherein performing the at least one mitigation action for the first pipe region comprises: conducting a field evaluation of the first pipe region, rehabilitating the first pipe region, replacing at least a portion of the first pipe region, generating a corrosion mitigation plan for the first pipe region, or any combination thereof.

15. The machine-readable storage medium of claim 12, wherein the microbial property comprises microbe type, microbe population, microbe growth data, substrate dependency, biocide efficacy, localization characteristics, or any combination thereof.

16. The machine-readable storage medium of claim 12, wherein simulating microbiologically induced corrosion within the first sampling segment using a microbiologically induced corrosion model further comprises simulating biofilm growth using a biofilm growth model, wherein a biofilm growth model output comprises the biofilm thickness, the biofilm density, or any combination thereof.

17. The machine-readable storage medium of claim 12, wherein the steps further comprise: displaying the microbiologically induced corrosion risk score and, optionally, the microbiologically induced corrosion risk profile, in a graphical user interface, wherein the graphical user interface comprises a geographic map that comprises a representation of the first pipe region localized to one or more locations on the geographic map, and wherein the microbiologically induced corrosion risk score is displayed as a color code overlaid on the representation of the first pipe region.

18. The machine-readable storage medium of claim 12, wherein the hydraulic profile comprises: a liquid phase in-situ velocity, a gas phase in-situ velocity, an oil density, a gas density, a water density, an oil viscosity, a gas viscosity, a water viscosity, an oil-water flow pattern, a gas-liquid flow pattern, a pressure, or any combination thereof.

19. The machine-readable storage medium of claim 12, wherein the likelihood criteria comprises: a production history, a leak history, a pipe coating composition, a pipe coating application history, a pipe coating location, a scraping compliance metric, biocide use data, or any combination thereof.

20. The machine-readable storage medium of claim 12, wherein calculating the microbiologically induced corrosion risk score comprises performing a statistical analysis using the microbiologically induced corrosion risk profile.
